(19) United States
(12) Patent Application Publication
(10) Pub. No.: US 2005/0112601 A1
(43) Pub. Date: May 26, 2005

(54) Methods for cellular or microorganism capture and quantification using bioluminescence regenerative cycle (BRC) assays

(76) Inventors: Arjang Hassibi, Palo Alto, CA (US); Siavash Ghazvini, Menlo Park, CA (US)

Correspondence Address: Edwin H. Taylor;Blakely, Sokoloff, Taylor & Zafman LLP, 12400 Wilshire Boulevard, 7th Floor, Los Angeles, CA, 90025, US

(21) Appl. No.: 10/788,579

(22) Filed: Feb. 27, 2004

Related U.S. Application Data
(60) Provisional application No. 60/451,107, filed on Feb. 27, 2003.

(51) Int. Cl. C12Q 1/68; G01N 33/554; G01N 33/569; C12M 1/34

(52) U.S. Cl. 435/6; 435/7.32; 435/287.2

(57) ABSTRACT

The methods, apparatus and compositions disclosed herein concern the detection, identification and/or quantification of target cells and/or microorganisms in samples. The assays are based on light emission detected from a bioluminescence regenerative cycle (BRC). Light emission may be related to cell and/or microorganism number through the number of ATP and PPi molecules per cell or microorganism. In certain embodiments of the invention, specific target cells and/or microorganisms may be separated from samples using one or more capture molecules, such as antibodies. The cells and/or microorganisms may be lysed, the contents purified in whole or in part and the ATP and PPi contents determined by BRC. Other embodiments of the invention concern apparatus comprising a series of chambers connected by a monodirectional flow channel, each chamber comprising an affinity matrix with one or more binding moieties attached. In certain embodiments, a multiplex assay may be performed using both antibodies and oligonucleotide probes specific for a pathogen of interest.

### BACKGROUND OF THE INVENTION

1. Field of the Invention

The present invention relates to the field of cell and/or microorganism detection, identification and/or quantification. More particularly, the present invention concerns novel approaches to detection, identification and/or quantification of cells and/or microorganisms, using a bioluminescence regenerative cycle (BRC) technique.

2. Description of Related Art

Various assays have been directed towards detection of contamination of surfaces, food, water and other substances by bacteria or other types of cells or microorganisms. Food or water supplies may need to be checked for contamination by cells or microorganisms that could cause disease if consumed. In the context of biowarfare, it may be desirable to test buildings, packages, letters or other items for contamination. In other cases, assays may be directed towards detection of specific types of cells, such as cancer or other diseased cells, in a sample from a patient. Ideally, such tests should detect, identify and/or quantify the microorganisms or cells present in a sample.

Standard tests for microorganisms or cells are typically based on either antibody assays or some variant of selective nucleic acid amplification. A large number of commercially available assays utilize antibody-based immunoassays, such as ELISA. An antibody or antibodies that bind selectively, preferably specifically to an antigen associated with the target cell or microorganism is exposed to a sample. Binding of target to antibody is detected by some type of label, e.g. a fluorescent probe or an enzyme that catalyzes the production of a colored dye. In different variations, the label may be attached to the primary antibody, to the target molecule, or to a secondary antibody that also binds to the target.

While antibody-based assays are of broad general utility, such tests may exhibit certain deficiencies. In particular, the sensitivity of detection may be too low to detect trace amounts of microorganisms or cells in a sample, which are still capable of causing disease if ingested or otherwise exposed to subjects.

Nucleic acid amplification assays, such as polymerase chain reaction (PCR®) amplification, are also commonly used for detection of target cells or microorganisms. In such assays, one or more primers that bind to nucleic acid sequences from the target of interest are added to a sample, along with a DNA polymerase and appropriate substrates and cofactors. The target nucleic acid sequence is replicated and detected by gel electrophoresis or other methods. Although PCR® and related techniques are generally more sensitive than immunoassays, they also suffer from various deficiencies. Many complex samples contain contaminants that may inhibit or otherwise interfere with the amplification reaction, making quantification difficult. In such complex samples, it may also be difficult to control the stringency of primer hybridization, allowing amplification of non-specific sequences to occur. There are a variety of circumstances in which nucleic acid amplification assays can produce false positive or false negative results. A need exists for an accurate, sensitive and robust method of detecting, identifying and/or quantifying microorganisms and/or cells in a wide variety of samples.

### SUMMARY OF INVENTION

The present invention addresses a long-felt need in the art by providing a novel assay system for detection, identification and/or quantification of microorganisms and/or cells. The system is capable of quantifying the amounts of ATP and pyrophosphate (PPi) present in target cells and/or microorganisms in samples. The ATP and PPi content are in turn correlated with the number of cells and/or microorganisms in the sample. The assay system is based on detection of chemiluminescence generated by a luciferin/luciferase linked reaction. In various embodiments of the invention, the sensitivity and/or stability of the system are greatly increased by using a bioluminescence regenerative cycle (BRC). The BRC assay can be adapted and used for many different detection methods. It is robust, simple and easy to use, with the capability of uniquely identifying low abundance microorganisms and/or cells from a wide variety of samples.

### DESCRIPTION OF ILLUSTRATIVE EMBODIMENTS

Terms that are not otherwise defined herein are used in accordance with their plain and ordinary meaning.

As used herein, “a” or “an” may mean one or more than one of an item.

As used herein, a “binding moiety” is a molecule or aggregate that has binding affinity for one or more target cells and/or microorganisms. Within the scope of the present invention virtually any molecule or aggregate that has a binding affinity for some target of interest may be a “binding moiety.” In preferred embodiments, the “binding moiety” is an antibody. In alternative embodiments the binding moiety may include, but is not limited to an aptamer, affibody, antibody fragment, humanized antibody, chimeric antibody, single-chain antibody, protein or peptide ligand, lectin or any other molecule or aggregate that can bind to one or more target cells or microorganisms. In preferred embodiments, the binding moiety is specific for binding to a single target cell or microorganism, although in other embodiments the binding moiety may bind to more than one targets that exhibit similar structures or binding domains.

Pyrophosphate-Based Luminescence Detection

The basic concept of assaying for pyrophosphate content using a luciferin/luciferase reaction was introduced by Nyren and others. (E.g., Nyren and Lundin, Anal. Biochem. 151:504-509, 1985.) This enzymatic detection method has been used for various applications, such as single nucleotide polymorphism (SNP) detection and DNA sequencing by synthesis (e.g., U.S. Pat. Nos. 4,971,903; 6,210,891; 6,258,568; 6,274,320). A coupled reaction occurs wherein pyrophosphate is generated by an enzyme-catalyzed process, such as nucleic acid polymerization. Pyrophosphate is used to produce ATP, in an ATP sulfurylase catalyzed reaction with adenosine 5′-phosphosulphate (APS). The ATP in turn is used for the production of light in a luciferin-luciferase coupled reaction.

Although the pyrophosphate-based system provides for convenient luminescent assays of use in a variety of biochemical or biological assays, the system provides insufficient sensitivity for detection of very low-level analytes, such as rare cells or microorganisms. An improved system for detection and quantification of low-level analytes is based on the bioluminescence regenerative cycle (BRC).

Bioluminescence Regenerative Cycle (BRC)

In general, the majority of cell or microorganism species present in a given environment will have an identical quantity of intracellular molecules. In particular, ATP and PPi content tend to be uniform for a particular type of cell or microorganism in a given environment. Thus, it is possible to determine the number of cells and/or microorganisms in a sample by measuring the content of ATP and PPi.

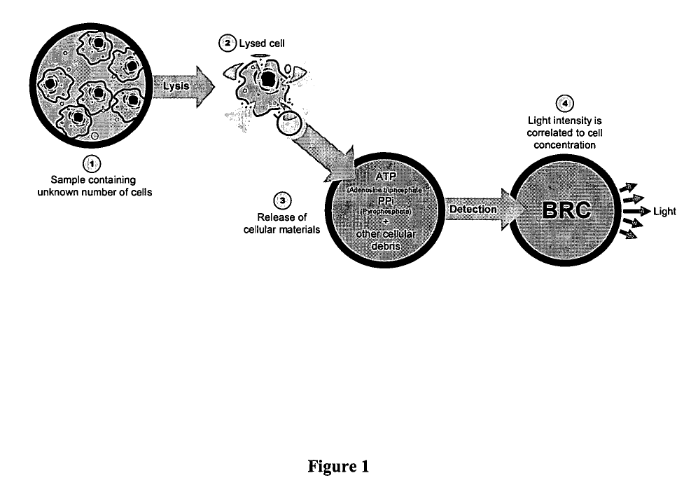

In various embodiments of the invention, the ATP and PPi content of samples are quantified using a bioluminescence regenerative cycle (BRC). In particular embodiments, cells and/or microorganisms of interest are separated from the rest of a sample using one or more binding moieties (e.g., antibodies). After lysis of the cells, the intracellular debris is released in the medium, for example by centrifugation, filtration or other known techniques. The total concentration of ATP and PPi is determined by BRC. The light intensity emitted during the BRC process is correlated to the enzymatic substrate (ATP and PPi) and consequently to the cell concentration, using the average amount of ATP and PPi present in each cell. The general scheme involved is illustrated in FIG. 1.

The bioluminescence regenerative cycle (BRC) is disclosed in more detail in U.S. patent application Ser. No. 10/186,455, filed Jun. 28, 2002, the entire text of which is incorporated herein by reference. BRC is novel method for quantifying the combined level of pyrophosphate (PPi) and adenosine-triphosphate (ATP) molecules. This assay does not require any molecular modification or labeling and merely implements a bioluminescence enzymatic reaction, activated by the presence of PPi and/or ATP molecules.

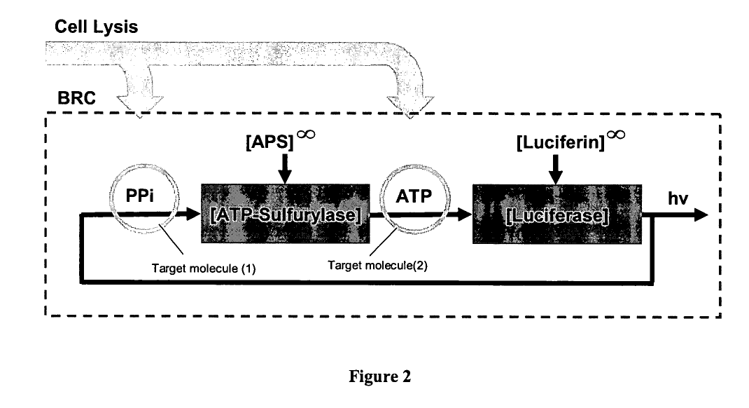

The regenerative cycle includes ATP-sulfurylase enzyme, which converts PPi to adenosine triphosphate (ATP) by consuming adenosine phosphosulfate (APS). The cycle also includes firefly luciferase and luciferin, which consume ATP as an energy source to generate photons as a signal. The luciferin/luciferase reaction yields AMP and PPi as products (FIG. 2). The PPi is recycled by ATP-sulfurylase to regenerate ATP (FIG. 2). In the course of the reactions, APS and luciferin are consumed and AMP and oxyluciferin are generated, while ATP sulfurylase and luciferase remain constant.

After each BRC cycle, a quantum of light is generated for each molecule of PPi and/or ATP in the original sample. Because the luciferase reaction is significantly slower than the ATP-sulfurylase reaction, in the presence of sufficient amounts of the substrates APS and luciferin a steady state cycle is maintained, in which the concentration of ATP and the resulting levels of light emission remain relatively constant for a considerable time. As a result, the photon emission rate remains steady and is a monotonic function of the amount of ATP and PPi in the initial mixture. For very low substrate concentrations (lower than 10⁻⁸ M), the total number of photons generated in a fixed time interval is proportional to the combined number of PPi and ATP molecules.

An advantage of the present invention is that the number of photons generated by the regenerative cycle can potentially be orders of magnitude higher than the initial number of PPi (or ATP) molecules introduced to the system. This results in greatly increased sensitivity of detection for longer integration times of detection.

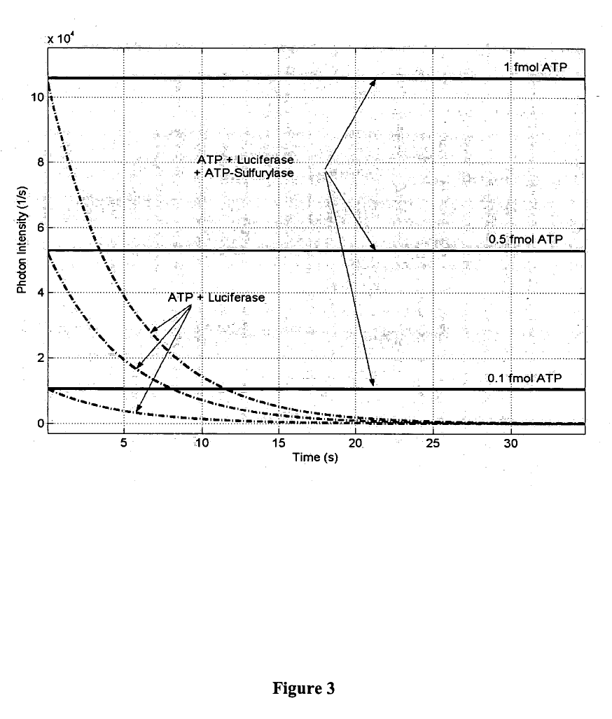

FIG. 3 illustrates an exemplary simulation of light emission for a BRC system, compared to a standard luminescent ATP assay with no enzymatic regenerative cycle. As indicated in the figure, the use of a regenerative cycle (BRC) stabilizes light intensity in contrast to the assay with no regenerative cycle, allowing photon accumulation over an extended time and greatly enhancing the sensitivity of detection.

Enzymatic Bioluminescence Cycle for PPi

To generate photons efficiently from pyrophosphate, the ATP-sulfurylase enzyme is used to catalyze the transfer of the adenylyl group from ATP to inorganic sulfate. The sulfurylase enzyme is ubiquitous in nature, although its physical role depends on the metabolic lifestyle of the organism. Here the enzyme is used to generate ATP from pyrophosphate by consuming adenosine phosphosulfate (APS):

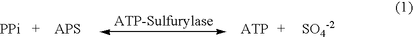

To complete the chemical process for light generation, firefly luciferase is used. This enzyme consumes the generated ATP to emit photons (λₘₐₓ=565 nm, Q.E.). This process uses luciferin as a substrate and generates oxyluciferin, adenosine monophosphate (AMP), CO₂ and PPi as byproducts.

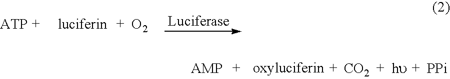

It is apparent from (1) and (2) that the PPi molecules generated at the end of the photon emission process by luciferase can again trigger the ATP synthesis reaction by ATP-sulfurylase, which results in a substrate cycling phenomenon (enzymatic positive feedback). Because this positive feedback regulates the total amount of ATP molecules in the solution, the light emission can also be regulated without any decay. The chemical yield of one PPi molecule per ATP from luciferase is close to unity; therefore this phenomenon may be modeled as an ideal unity-gain positive feedback system. This positive feedback regulates the process and prevents any drop in light generation due to substrate consumption.

Bioluminescenec Super Regenerative Cycle (BSRC) Assays

In certain embodiments of the invention, an additional enzymatic complex may be added to the standard BRC reaction: Adenylate Kinase (AK) in the presence of AMP substrate, and pyruvate kinase (PK) in the presence of phosphoenolpyruvate (PEP). The additional enzymes can create two ATP molecules from a single ATP by substrate cycling. This process would exponentially increase the concentration of ATP molecules in the reaction buffer. Since bioluminescence light activity of luciferase is proportional to the ATP concentration, the amount of light generated grows exponentially as a function of time. The rate of light generation growth depends on the kinetics of AK and PK and the concentration of their substrates.

The light intensity generated in this BSRC process, considering an exponential growth rate of k for ATP molecules is a function of time defined by

$$I = ( \frac{α \cdot {k}_{L}}{V} ) \cdot [ {N}_{ATP} + {N}_{PPi} ] \cdot exp ( kt ) \qquad ( 3 )$$

This assay generates more photons compared to Normal BRC. However, the quantifying of the original concentration of PPi, ATP or the combination of PPi and ATP necessitates kinetics analysis, in contrast to normal BRC, which is time insensitive.

Detection of Cells or Microorganisms by BRC

Sample Isolation

In various embodiments of the invention, samples suspected of containing one or more microorganisms and/or cells may be collected and processed. Sample processing may be used, for example, to remove contaminants that could interfere with the BRC process by light quenching, enzyme inhibition, etc. The embodiments are not limiting as to the type of sample that may be analyzed, and samples may include without being limited to blood, serum, plasma, cerebrospinal fluid, lymphatic fluid, urine, stool, semen, lacrimal fluid, saliva, sputum, a biopsy sample, a tissue scraping, a swab sample, an endoscopic sample, a cell sample, a tissue sample, food, water, environmental swab samples, air samples and any other sample that could potentially contain cells and/or microorganisms. Samples may be initially processed using any of a variety of known procedures, such as homogenization, extraction, enzymatic digestion (e.g., protease, nuclease), filtration, organic phase extraction, centrifugation, ultracentrifugation, column chromatography, HPLC, FPLC, electrophoresis or any other type of known sample preparation, without limitation. In various applications, it may be appropriate to separate a sample into specific components, such as separating a blood sample into a cellular component and a serum component. In preferred embodiments, the final prepared sample to be analyzed will comprise an aqueous preparation with possible known or unknown cells and/or microorganisms.

Target Isolation Using Capture Molecules

In particular embodiments of the invention, it may be appropriate to use one or more binding moieties to capture specific types of cells and/or microorganisms, such as infectious pathogens. A variety of such binding moieties may be used, including but not limited to polyclonal antibodies, monoclonal antibodies, antibody fragments, chimeric antibodies, affibodies, aptamers, protein ligands, or any other known binding moiety. Such capture agents may be purchased from a wide variety of commercial sources, or may be generated using methods well known in the art (e.g. U.S. Pat. Nos. 5,270,163; 5,567,588; 5,670,637; 5,696,249; 5,843,653; Harlowe and Lane, Antibodies: A Laboratory Manual, Cold Spring Harbor Press, Cold Spring Harbor, N.Y., 1988).

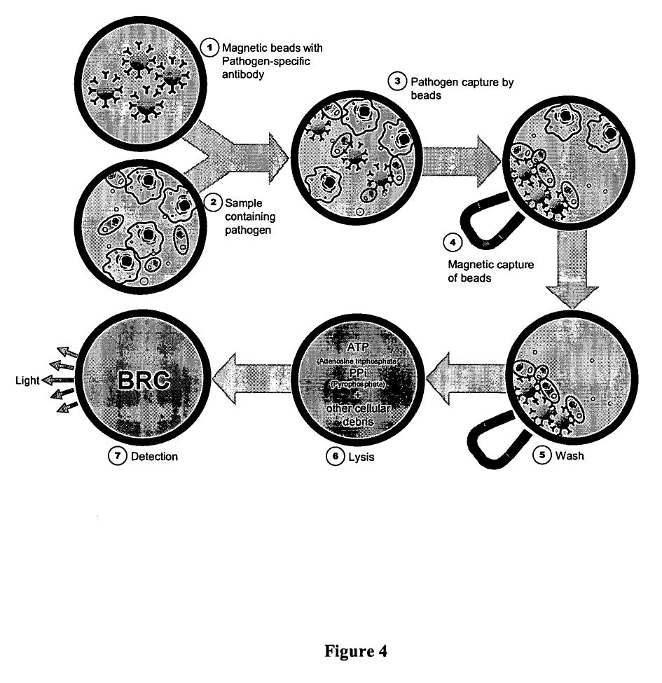

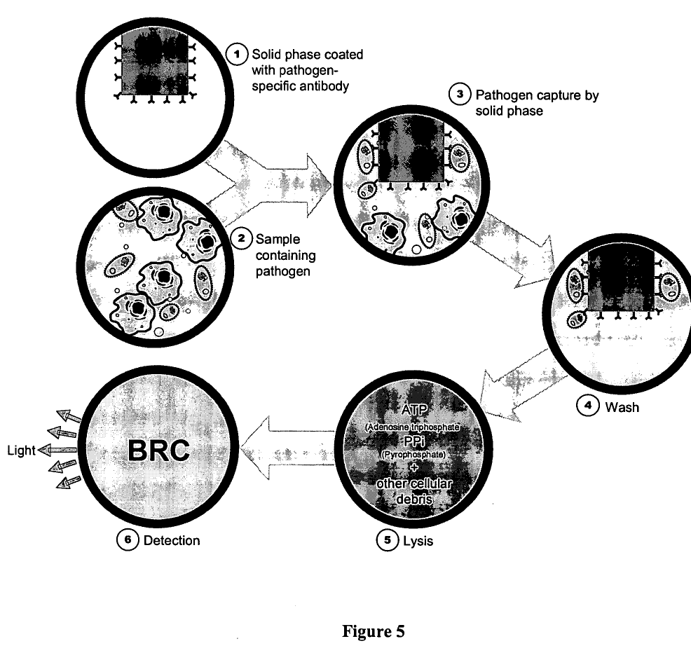

For example, in applications involving human pathogen profiling tests, one or more pathogen specific antibodies may be used to capture the target. Immunoaffinity methods suitable for target separation are known in the art, including but not limited to use of antibody-conjugated magnetic beads, attachment to glass or plastic beads and FACS (fluorescent activated cell sorter) attachment of antibodies to solid supports such as nitrocellulose or nylon membranes, or use of various affinity matrices (FIG. 4 and FIG. 5). Alternatively, the total number of cells or microorganisms in a given sample may be determined, in which case separation of specific targets is not necessary.

Lysis

To facilitate separation of ATP and PPi from other cellular components, cell lysis may or may not be used. A variety of methods of lysis are known, including but not limited to homogenization, detergent solubilization, protease treatment, sonication and/or use of various apparatus such as Waring blenders or Virtis homogenizers. Any known technique that does not degrade ATP and/or PPi may be used.

BRC Assay

The BRC assay is used to quantify the sample concentration of target cells and/or microorganisms. Quantitative analysis relies upon the relationship between the number of cells and/or microorganisms in a sample and the light intensity detected by the assay. Assuming that there are a regulated and fixed number of ATP molecules, N_{ATP}, and PPi, N_{PPi}, in each cell then the total number of detectable substrate molecules for BRC assay per cell, N_{Cell} is
N_{Cell}=N_{ATP}+N_{PPi} (4)

Since the photon generation process of BRC is only a function of the turnover of luciferase, rather than ATP-sulfurylase, the simplified equation expressing light intensity I, is

$$I = α \cdot \frac{ⅆ}{ⅆ t} ( \frac{{N}_{ATP}}{V} ) = ( \frac{α \cdot {k}_{L}}{V} ) \cdot {N}_{Sub} ( t ) , \qquad ( 5 )$$

or

$$I = ( \frac{α \cdot {k}_{L}}{V} ) \cdot {( {N}_{Sub} )}_{0} . \qquad ( 6 )$$

where V is the volume of the reaction buffer, k_{L} the turnover rate of luciferase, a the quantum efficiency of the bioluminescence process, and (N_{Sub})₀ the initial quantity of BRC substrates (PPi and ATP) in the reaction buffer volume. With X number of cells in the sample, the light intensity based on (4) and (6) is

$$I = ( \frac{α \cdot {k}_{L}}{V} ) \cdot X \cdot {N}_{Cell} , \qquad ( 7 )$$

Thus, the light intensity out of the assay is in fact proportional to the cell count. As an example if there are 10⁶ substrate molecules per cell, then in order to assess the cell count from an assay, emitting I_{X} photons per second per unit volume, the following relationship would apply.

$$X = \frac{{I}_{X}}{( \frac{α \cdot {k}_{L}}{V} ) \cdot {N}_{Cell}} = \frac{{I}_{X}}{( \frac{α \cdot {k}_{L}}{V} ) \cdot {10}^{6}} , \qquad ( 8 )$$

As disclosed herein, the BRC assay may be used to accurately quantify the number of target cells and/or microorganisms present in a sample, based on the emitted light intensity. Accurate estimates of cell and/or microorganisms number will be based on estimates of the amount of ATP and PPi per cell or microorganism. As the skilled artisan will appreciate, a variety of methods are available to derive such estimates. For example, target cells and/or microorganisms may be isolated from a given sample and the number of cells counted by a variety of known techniques, such as cell sorting by FACS, microscopic estimates of cell number, etc. The sample, containing a known number of cells, may then be subjected to BRC assay and the light emission quantified. Using such techniques, the number of cells and/or microorganisms in a new sample may be determined simply based on the relationship of BRC emitted light per unit cell, without separately quantifying ATP and PPi. Alternatively, the ATP and PPi content per cell or microorganism may be determined by chemical analysis or may be obtained from reported values in the literature. The light emission from BRC may be quantified using known amounts of ATP and/or PPi standard solutions. Light emission from a new sample may then be related to ATP plus PPi content and the cells quantified.

Detection Systems

Photons generated by the enzymatic process are counted using BRC to estimate the quantity of the cells and/or microorganisms. Generation of photons by luciferase has a quantum efficiency (Q.E.) of approximately 0.88 per consumed ATP molecule. The maximum wavelength (dependent upon the particular type of luciferase) is in the visible range of the optical spectrum (e.g., 565 nm for firefly luciferase).

A variety of photosensitive devices developed to detect bioluminescent signals may be used for detecting light from the BRC assay. These devices include photomultiplier tubes (PMTs), charge coupled devices (CCDs), and photodiodes. The photosensitive device can either be in close proximity to the BRC reaction to receive the incident photons directly, or at a distance from the reaction buffer with a light coupling device (e.g. optical fiber or mirror system) to convey photons from the sample to the detector.

In an exemplary embodiment of the invention, the detection system may comprise a cooled CCD camera imaging system (IVIS; Xenogen) or a luminometer (Lmax™; Molecular Devices) that employs a single PMT detector. The light coupling efficiencies of each system (including path loss), from the microarray is approximately 0.012% for the CCD and 8% for the PMT systems. The photosensitive device is typically either in direct proximity of the BRC reaction to directly receive incident photons, or relatively far from the buffer with a light coupling device (e.g. optical fiber or mirror system) capable of directing light from the sample to the detector. In an exemplary embodiment, a UDT-PIN-UV-50-9850-1 photodiode (Hamamatsu Corp., Hamamatsu, Japan) was used with a transimpedance amplifier with a gain of 10⁸ volts/amp.

In addition to standard imaging systems, other alternative detectors comprising photodiode systems may be used to detect the generated photons from the assay. Although the performance of single photodiode systems is inferior to PMT systems or CCD image sensors because of the steady light intensity of BRC, in particular applications the single photodiode system may result in equivalent performance. The cost of and the physical size of the detector can be reduced extensively using a single photodiode system.

### EXAMPLES

### Example 1

### Detection of Cells and/or Microorganisms by BRC

Assay Conditions

In an exemplary embodiment of the invention, the BRC assay may be performed in 50 μl of reaction mixture (see Ronaghi et al., Anal. Biochem. 242:84-89, 1996 with modifications) containing 250 ng luciferase (Promega, Madison, Wis.), 50 mU ATP sulfurylase (Sigma Chemical Co., St. Louis, Mo.), 2 mM dithiothreitol, 100 mM Tris-Acetate pH 7.75, 0.5 mM EDTA, 0.5 mg BSA, 0.2 mg polyvinylpyrrolidone (Mr 360.000), 10 μg D-luciferin (Biothema, Dalaro, Sweden), 5 mM magnesium acetate and 10 attomole to 0.01 attomole purified pyrophosphate or ATP. The addition of very low amounts of pyrophosphate or ATP (or analogs) may act to decrease background light emission from the reaction mixture. The generated light intensity over a time interval may be used to calculate the number of target cells and/or microorganisms in the sample.

Apparatus

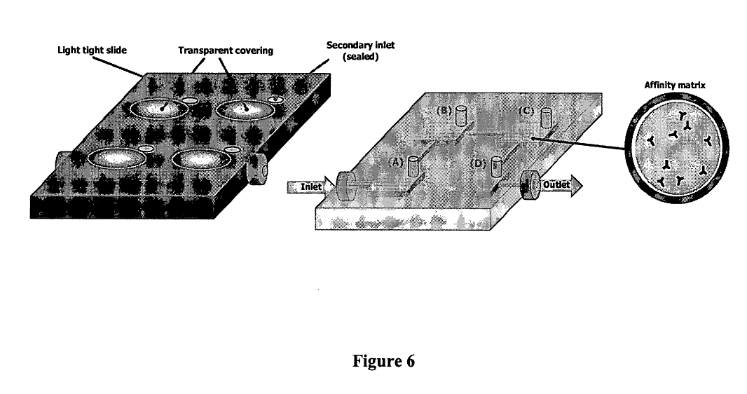

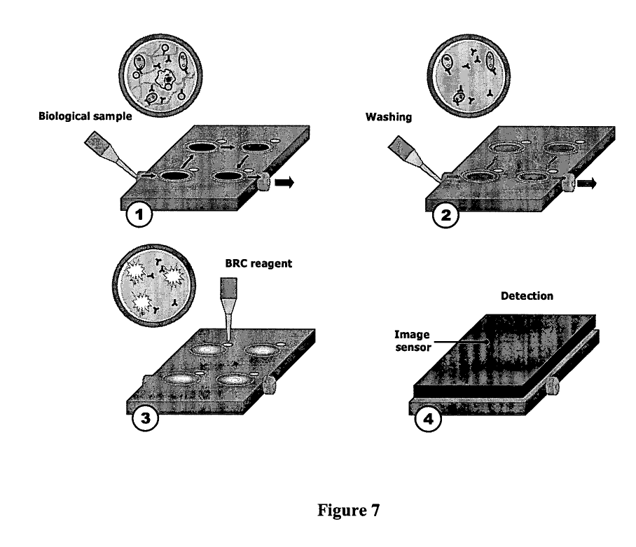

Another exemplary embodiment of the invention, illustrated in FIG. 6 and FIG. 7, concerns an apparatus comprising a plurality of chambers, connected via a mono-directional flow through system in a cassette. In this apparatus, each sealed chamber contains a specific affinity matrix for capturing a specific biological entity. As a biological sample of interest is moved through a flow channel, the sample is extensively exposed to all capture site, allowing target capture to occur. After the capture phase, additional washing procedures may be performed using the same flow channel system to get rid of the extracellular debris or unwanted biological background molecules in the sample. Next, lysis reagents and potentially BRC detection assay reagents may be added individually to the chambers. This may be carried out by injecting the reagents using secondary inlets, for example by breaking the initial seals on the inlets. The cassette may be inserted or incorporated into an imaging system that individually measures the photon flux from each chamber, hence quantifying any captured microorganisms.

The methods described herein may be used to detect, identify and/or quantify a variety of pathogens. For example, oligonucleotide probes specific for pathogen DNA may be immobilized in the chambers of the apparatus disclosed above. Alternatively, immunoassays using one or more antibodies specific for a pathogenic protein or other antigen may be performed in the apparatus. Where very high specificity detection is needed, it is possible to perform multiplex assays using an antibody capture of the pathogen in combination with DNA detection of the same pathogen.

In an exemplary method, a biological sample may be processed through the cassette, with each chamber containing a microfibrous material or beads with attached antibodies specific for a pathogen of interest. The same chambers may also contain one or more oligonucleotide capture probes specific for the pathogen nucleic acid. Once the pathogen has been captured in the chamber by the antibody, the pathogen may be lysed and pathogen nucleic acids detected through hybridization to the oligonucleotide probe(s). Thus, detection is dependent upon two independent binding events, one of an antibody to a pathogen antigen and a second of an oligonucleotide to a pathogen nucleic acid.

As disclosed above, pathogens may be quantified by detection of ATP and PPi after pathogen lysis. Once ATP and PPi have been quantified, the chambers may be washed and bound nucleic acids may be detected using standard BRC assays, as disclosed in U.S. patent application Ser. No. 10/186,455, filed Jun. 28, 2002. Alternatively, hybridized nucleic acids may be detected using a branched BRC method, as described in Provisional U.S. Patent Application Ser. No. 60/440,670, filed Jan. 15, 2003, the entire contents of which are incorporated herein by reference.

A multiplexing assay can also be performed with beads in solution. Two different types of beads may be used, one attached to an antibody against the pathogen and the other attached to an oligonucleotide probe specific for a pathogen nucleic acid. Both beads may be mixed with blood or other samples, the beads may be captured, and the cells lysed the cells. Cells may be quantified using BRC detection of PPi and ATP. The released nucleic acid will also bind to the oligonucleotides on the other type of bead in solution. Once ATP and PPi have been quantified, the beads may be washed and the hybridized nucleic acids detected, for example by a branched BRC assay. Use of such multiplexed assays allows the validation and typing of the strain(s) of pathogen present in a sample.

All of the COMPOSITIONS, METHODS and APPARATUS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS, METHODS and APPARATUS and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1) A method comprising:
a) obtaining one or more samples;
b) obtaining one or more types of target cells and/or microorganisms from each sample;
c) performing a bioluminescence regenerative cycle (BRC) assay to detect ATP and inorganic phosphate (PPi) in the cells and/or microorganisms; and
d) estimating the number of target cells and/or microorganisms from the BRC assay.

2) The method of claim 1, further comprising using one or more binding moieties to separate one or more types of target cells and/or microorganisms from the sample.

3) The method of claim 2, wherein the capture moiety is an antibody.

4) The method of claim 3, wherein the antibody is attached to a magnetic bead, glass bead, plastic bead, nitrocellulose membrane, nylon membrane, chromatography support or other solid surface.

5) The method of claim 1, further comprising lysing the cells and/or microorganisms.

6) The method of claim 5, further comprising partially purifying the ATP and PPi from the lysed cells and/or microorganisms.

7) The method of claim 1, wherein the sample is blood, serum, plasma, cerebrospinal fluid, lymphatic fluid, urine, stool, semen, lacrimal fluid, saliva, sputum, a biopsy sample, a tissue scraping, a swab sample, an endoscopic sample, a cell sample, a tissue sample, a food sample, a water sample, an environmental sample or an air sample.

8) The method of claim 1, further comprising accumulating the photon count over a time interval.

9) The method of claim 1, further comprising adding adenylate kinase and AMP substrate or pyruvate kinase and phosphoenolpyruvate to the BRC assay.

10) The method of claim 1, further comprising adding between 0.01 and 10 attomole of purified pyrophosphate or ATP to each assay to reduce background light emission.

11) A kit for performing BRC assays comprising:
a) one or more BRC reagents; and
b) a standard solution.

12) The kit of claim 11, wherein the standard solution comprises a known amount of ATP and/or PPi.

13) The kit of claim 11, wherein the BRC reagents comprise at least one reagent selected from the group consisting of luciferin, luciferase, ATP sulfurylase, adenosine 5′-phosphosulphate and BRC buffer.

14) An apparatus comprising:
a) a light tight slide;
b) a plurality of chambers in the slide, the chambers connected by a mono-directional flow channel;
c) an inlet and an outlet connected to the flow channel; and
d) a sealed secondary inlet connected to each chamber.

15) The apparatus of claim 14, further comprising an affinity matrix in each chamber.

16) The apparatus of claim 15, wherein each affinity matrix is attached to at least one binding moiety.

17) The apparatus of claim 16, wherein the binding moieties are antibodies or capture oligonucleotides.

18) The apparatus of claim 17, wherein each chamber comprises at least one antibody and at least one capture oligonucleotide, both specific for the same pathogen.
